(19) United States
(12) Patent Application Publication
(10) Pub. No.: US 2013/0131803 A1
(43) Pub. Date: May 23, 2013

(54) LIGAMENT AND TENDON PROSTHESIS MADE FROM CABLES OF FILAMENTS

(71) Applicant: Idan Tobis, Beit Hashmonai (IL)

(72) Inventor: Idan Tobis, Beit Hashmonai (IL)

(73) Assignee: TAVOR [I.T.N] LTD., Ashqelon (IL)

(21) Appl. No.: 13/814,655

(22) PCT Filed: Aug. 9, 2011
(86) PCT No.: PCT/IL11/00647

§ 371 Date: Feb. 6, 2013

Related U.S. Application Data
(60) Provisional application No. 61/371,766, filed on Aug. 9, 2010.

(51) Int. Cl. A61F 2/08

(52) U.S. Cl. CPC A61F 2/08
USPC 623/13.19

(57) ABSTRACT

The invention provides a ligament or tendon prosthesis made from two or more of cables braided into a helical structure, where each cable is made from two or more strands. The strands may be made from an alloy exhibiting pseudoelastic properties at body temperature, and may be twisted into a helical structure in the cables.

### FIELD OF THE INVENTION

This invention relates to medical devices, and more specifically to such devices for treating a ruptured, torn or otherwise damaged ligament or tendon.

### BACKGROUND OF THE INVENTION

The human and animal body contains numerous tendons and ligaments. Although tendons and ligaments have similar anatomical structures, they serve different biological functions. Both serve as load-bearing structures, with tendons joining muscle to bone, while ligaments join bone to bone.

Ligament and tendon injuries are very common in sports that require rapid stopping and starting or quickly changing directions. Under such conditions, the extreme forces on the knee, for example, can result in torn ligaments. The anterior cruciate ligament (ACL) and the medial collateral ligament (MCL) are the most often injured, but the posterior cruciate ligament (PCL) and the lateral collateral ligament (LCL) can also be injured.

One method for repairing a torn tendon or ligament is to fasten the cut ends of the tendon or ligament together by means of a suture. In the case of more extensive injuries where loss of substance must be bridged, a known method of treatment involves transplanting a graft consisting of a ligament or tendon harvested either from another location in the patient's body (an autograft) or from a donor (allograft). Although this process is often successful, it results in loss of some degree of mobility in the donor location, as well as various other complications such as pain and local morbidity at the donor site in the case of an autograft, and a risk of infection in the case of an allograft.

Another method for treating a torn ligament or tendon is to introduce a prosthesis to replace the torn ligament or tendon. Such prostheses are typically formed as a bundle of loosely bundled fibers or a coreless tubular structure.

The material of the prosthesis should be compatible with other body tissues and at the same time the prosthesis should be able to resist the abrasion that occurs when the bone rubs against the surface of the prosthesis during movement. In recent years, prostheses of materials without adverse tissue reaction, such as Dacron, Teflon and polypropene, have been used for replacing tendons and ligaments (including the ACL). While it has been possible to reduce the time that the patient must keep the body part to which the prosthesis is attached immobilized, these prostheses sometimes result in the formation of granuloma and incomplete restoration of function. In particular, for cruciate ligament prostheses, it is difficult to obtain either a satisfactory stability of the knee joint or the necessary strength of the tissue formed upon healing, and after prolonged use the prostheses sometimes break due to fatigue of the material.

Patent Publication WO 2010/134943 discloses a device comprising a degradable material and biocompatible non-degradable polymeric fiber-based material, in a three-dimensional braided scaffold. End sections are designed to allow bone cell ingrowth, and one or more middle regions are designed to allow ligament or tendon cell ingrowth.

European Patent Publication EP 106501 discloses a ligament or tendon prosthesis having multiple longitudinally parallel strands of microporous expanded polytetrafluoroethylene. Strand dimensions and microstructure are selected so that tissue can penetrate throughout. The prosthesis is formed from multiple loops of a single continuous strandThe strands are twisted or arranged in a loose braid about the prosthesis axis for improved load distribution during bending of the prosthesis.

European Patent Publication EP 0145492 discloses a replacement or augmentation for a damaged ligament or tendon comprising a multiplicity of flexible strands held together in a substantially parallel array. The strands form a braided sheathing over at least one portion.

U.S. Pat. No. 4,983,184 discloses an artificial soft tissue part and/or reinforcing a natural soft tissue part, such as a ligament or a tendon, having bundles of metal fibers held loosely together. The fibers preferably consist of an alloy based on titanium, and can be provided with a coating consisting of an organic substance resorbable in the body.

### SUMMARY OF THE INVENTION

The present invention provides a ligament or tendon prosthesis for use in replacing a torn or otherwise damaged, ligament or tendon, such as an anterior cruciate Ligament (ACL). The prosthesis of the invention may be used to replace a ligament or tendon in a human or any animal such as a horse, dog, cat, pig, or cattle.

The prosthesis of the invention comprises two or more helical cables braided together to form a braided structure. Each cable of the prosthesis comprises two or more strands that are twisted together. The strands may be made from a pseudoelastic alloy (also known as a “superelastic alloy”). such as an implantable Nitinol conforming to the ASTM Standard 2063. As shown below, strands made from a superelastic alloy endow the prosthesis with mechanical properties particularly suitable to replace a torn ligament or tendon.

The shape, elongation ranges, stiffness and ultimate load values of the prosthesis are preferably selected in order to match the tendon or ligament the prosthesis is designed to replace.

The prosthesis of the invention tends to have a relatively low resistance to bending and twisting movements as compared to the tensile resistance of the prosthesis, in comparison to a regular braid of solid wires of the same diameter. This allows implanting the prosthesis as a replacement for intra articular ligaments or tendons, which undergo extensive twisting and bending as compared to extra articular ligaments or tendons.

The invention thus provides a ligament or tendon prosthesis comprising two or more of cables braided into a helical structure, each cable comprising two or more strands. The prosthesis may further comprise an attachment device at one or both ends configured to allow an end of the prosthesis to be attached to bone. The prosthesis may have a hollow circular cross section.

Each cable may have a helical shape in the prosthesis, and may have, for example, a radius of 2-8 mm. The prosthesis may have a number of cables is in a range from 24 to 96, and the cables may be connected at points of contact between the cables in the braided structure.

The strands of the prosthesis may be made, for example, of an alloy exhibiting pseudoelastic properties at body temperature. The strands may be twisted together. The number of strands in a cable may be in a range from 3 to 19. The strands may be made from a pseudoelastic alloy such as Nitinol. Each strand in a cable may have a helical shape having a radius, for example, of 0.2-0.6 mm. The helical shape of a strand may have a ratio of radius to pitch that is smaller than a ratio of radius to pitch of cables having a helical shape in the prosthesis.

The prosthesis of the invention may have a non-linear force-elongation relationship.

In an embodiment of the invention, the prosthesis of the invention comprises 48 cables, each cable being 0.5 mm thick and comprising 19 strands, 0.1 mm thick, made of Nitinol with an austenitic final temperature of 25° C. The prosthesis may have a 3 mm elongation in a toe region, a 2.4 mm elongation in the pseudoelastic region, and an ultimate tensile load of 750N.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1a shows a prosthesis for replacing a ligament or tendon in accordance with one embodiment of the invention;

FIG. 1b shows the braided structure of the prosthesis of FIG. 1a;

FIG. 2 shows a cable from the prosthesis of FIGS. 1a and 1b comprising a plurality of strands;

FIG. 3 shows a model representing the nonlinear force/elongation relationship of the prosthesis of FIGS. 1a and 1b; and

FIG. 4 shows the force/elongation curve of the prosthesis of FIGS. 1a and 1b.

### DESCRIPTION OF EMBODIMENTS

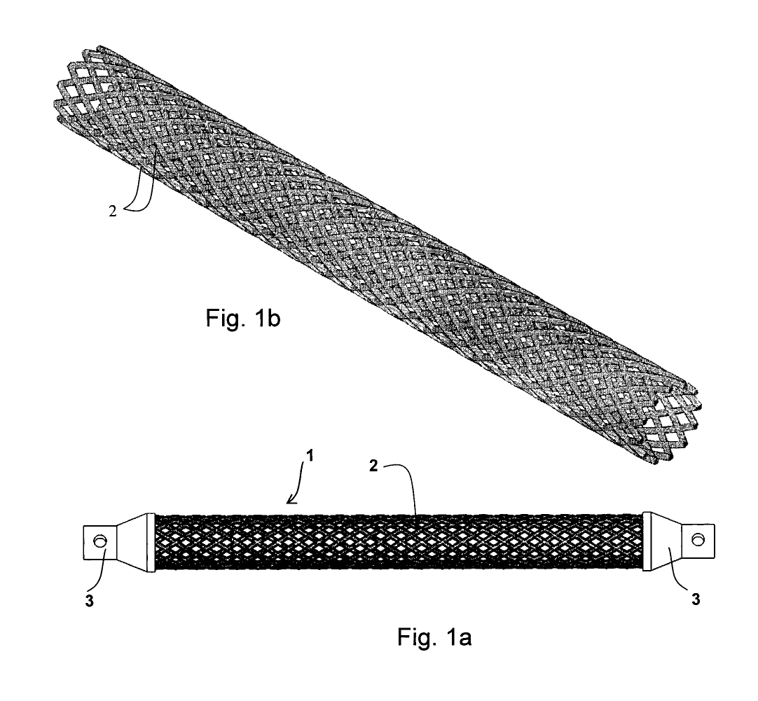

FIGS. 1a and 1b show a ligament or tendon prosthesis 1 in accordance with one embodiment of the invention. The prosthesis 1 is generally elongated in shape and has an attachment device 3 at one or both ends that is configured to allow an end of the prosthesis 1 to be attached to bone. As shown in greater detail in FIG. 1b, the prosthesis 1 comprises two or more helically shaped cables 2 braided together to form a braided structure having a hollow circular cross section. The number of the cables 2 in the prosthesis 1 may be, for example, from 24 to 96. The cables 2 may be loosely connected at points of contact between the cables in the braided structure.

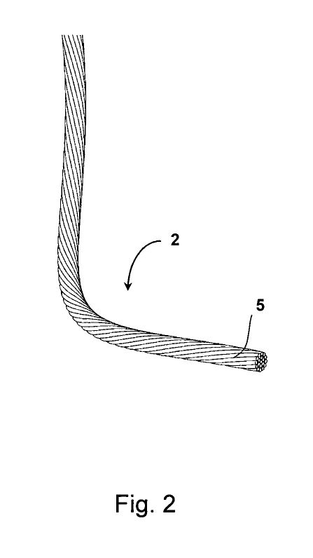

FIG. 2 shows the structure of a cable 2 of the prosthesis 1. Each cable 2 of the prosthesis 1 comprises two or more strands 5 that are twisted together into a second helical structure. The number of strands 5 in the cable 2 can be, for example, 3, 7, or 19. The strands 5 may be made from a pseudoelastic alloy such as an implantable Nitinol conforming to the ASTM Standard 2063. As shown below, strands 5 made from a superelastic alloy endow the prosthesis 1 with mechanical properties particularly suitable to replace a torn ligament or tendon.

Both the cables 2 and the filaments 5 have a helical structure, but with a different helix radius. The helix of the cables 2 may have a radius, for example, of 2-8 mm, depending on the ligament it is intended to replace, while the strands 5 may be twisted into a helix having a radius of 0.2-0.6 mm. The ratio of radius to pitch of the helices of the strands 5 in the cables structure is preferably is smaller than that of the cables 2 in the helical structure of the prosthesis.

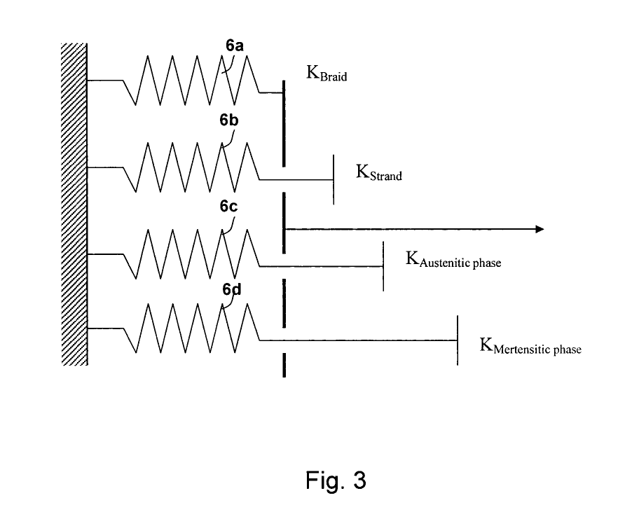

When the alloy is pseudoelastic, the ensuing force-elongation relationship is non-linear, with a different elastic modulus in the martensitic phase, transitional phase (“upper plateau”), and austenitic phase. In this case, as shown in FIG. 3, the mechanical properties of the prosthesis can be represented by four springs arranged in parallel: a spring 6a representing the cables 2 having a Young's modulus K_{strand}, a spring 6c representing the alloy in the austenitic phase having a Young's modulus K_{austenitic phase}, and a spring 6d representing the alloy in the martensitic phase having a Young's modulus K_{martensitic phase}.

When the prosthesis 1 is elongated, first the pitch of the cables 2 in the braided structure increases until it can no longer do so. At this point, the pitch of the strands 5 in the cables 2 begins to increase. Since the ratio of radius to pitch of the helices of strands 5 is smaller than that of the cables 2 in the braided structure, the stiffness of the strands 5 is larger than that of the braided structure. When the pitch of both the braided structure and strands are maximal, the alloy, which has a higher stiffness than that of the strands, begins to stretch.

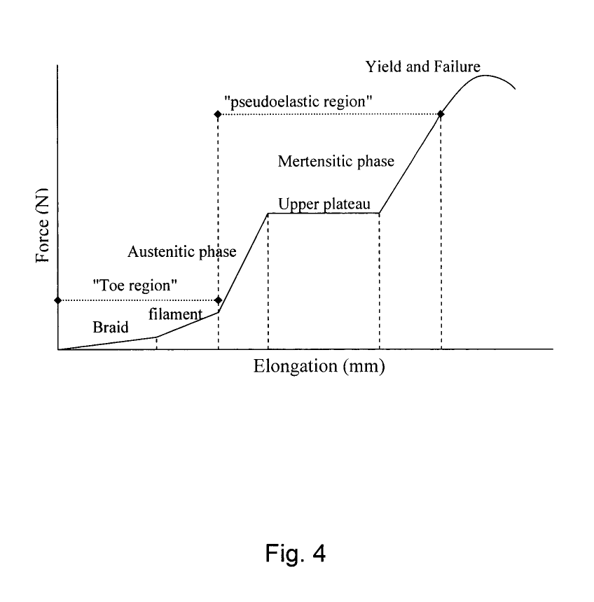

FIG. 4 illustrates the nonlinear force/elongation curve of the prosthesis, having two distinct regions: a toe region and pseudoelastic region. The toe region is where the helical structure of the cables, which has the lowest elastic coefficient, unwinds. Then, in the “strand” region, the helix of the strands 5, having a slightly higher elastic coefficient, unwinds. When further elongation is applied, beyond the ability of helical structure and the helices of the strands 5 to unwind, the alloy stretches pseudoelastically, which overall has the highest elastic coefficient. In the pseudoelastic region, the alloy is initially in its austenitic phase, but as elongation of the prosthesis continues, the alloy, after passing through an upper plateau region enters its martensitic phase.

Thus, for instance, a prosthesis designed to replace an anterior cruciate ligament in a male, might have a circular cross sectional braided structure comprising 48 cables, each cable being 0.5 mm thick and comprising of 19 strands, 0.1 mm thick, made of Nitinol with an austenitic final temperature of 25° C. This structure would allow a 3 mm elongation in the toe region, a 2.4 mm elongation in the pseudoelastic region, and has an ultimate tensile load of 750N.

1.-16. (canceled)

17. A ligament or tendon prosthesis, comprising:
two or more cables braided into a helical structure, each of the two or more cables comprising two or more strands.

18. The prosthesis according to claim 17 wherein the two or more strands are made of an alloy exhibiting pseudoelastic properties at body temperature.

19. The prosthesis according to claim 17, further comprising an attachment device at one or both ends configured to allow an end of the prosthesis to be attached to bone.

20. The prosthesis according to claim 17 wherein the helical structure defines a hollow, circular cross-section.

21. The prosthesis according to claim 17 wherein the two or more cables includes a number of cables in a range from 24 to 96.

22. The prosthesis according to claim 17 wherein the two or more cables are connected at points of contact therebetween in the braided structure.

23. The prosthesis according to claim 17 wherein the two or more strands are twisted together.

24. The prosthesis according to claim 17 wherein a number of the two or more strands in a respective cable of the two or more cables is in a range from 3 to 19.

25. The prosthesis according to claim 17 wherein the two or more strands are made from a pseudoelastic alloy.

26. The prosthesis according to claim 25 wherein the pseudoelastic alloy includes Nitinol.

27. The prosthesis according to claim 17 wherein each of the two or more cables has a helical shape having a radius of about 2 mm to about 8 mm.

28. The prosthesis according to claim 17 wherein each of the two or more strands in a respective cable of the two or more cables has a helical shape having a radius of about 0.2 mm to about 0.6 mm.

29. The prosthesis according to claim 28 wherein the helical shape has a ratio of radius to pitch that is smaller than a ratio of radius to pitch of cables having a helical shape in the prosthesis.

30. The prosthesis according to claim 17 wherein the two or more strands are made from a pseudoelastic alloy having a non-linear force-elongation relationship.

31. The prosthesis according to claim 17 wherein the two or more cables includes 48 cables, each of the two or more cables is about 0.5 mm thick and includes 19 wires that are about 0.1 mm thick and made of Nitinol having an austenitic final temperature of 25° C.

32. The prosthesis according to claim 17 having a 3 mm elongation in a toe region, a 2.4 mm elongation in the pseudoelastic region, and an ultimate tensile load of 750N.
